(19) United States
(12) Patent Application Publication
(10) Pub. No.: US 2024/0252754 A1
(43) Pub. Date: Aug. 1, 2024

(54) SINGLE-USE NEUROTOXIN FORMULATIONS AND PACKAGING

(71) Applicant: AEON Biopharma Sub Inc., Irvine, CA (US)

(72) Inventors: Adelbert L. Stagg, Irvine, CA (US); Andrew M. Blumenfeld, Del Mar, CA (US)

(21) Appl. No.: 18/682,352

(22) PCT Filed: Aug. 11, 2022
(86) PCT No.: PCT/US2022/074844

§ 371 Date: Feb. 8, 2024

Related U.S. Application Data
(60) Provisional application No. 63/232,410, filed on Aug. 12, 2021.

(51) Int. Cl. A61M 5/178; A61K 38/48

(52) U.S. Cl. CPC A61M 5/178; A61K 38/4893; A61M 2202/0007; A61M 2202/07; A61M 2205/584

(57) ABSTRACT

Disclosed herein are systems and devices for use with neurotoxin formulations.

### FIELD

The present specification relates to single-use packaging and formulation of pharmaceutical formulations.

### BACKGROUND

Current neurotoxin packaging and formulations are not geared toward specific clinical uses. For example, the FDA-approved Onabotulinumtoxin A treatment for chronic migraine comprises a fixed-site, fixed-dose (FSFD) protocol that includes 31 5-unit injections into 7 muscle regions for a total of 155 units. However, BOTOX® is supplied in 50-Unit, 100-Unit, and 200-Unit vials. As a result, certain treatments are completed with neurotoxin composition left in the vial, and clinicians are discouraged from using residual composition on a different patient.

In addition, materials used in neurotoxin packaging can impair the ability of practitioners to withdraw all of the neurotoxin from the packaging vessel, typically a glass vial.

These factors result in waste of toxin formulations as well as increased expense to clients. Therefore, improved packaging systems are desired.

### SUMMARY

Disclosed herein are systems, devices, formulations, kits, and methods for packaging neurotoxins, for example Clostridial neurotoxins, including botulinum toxins. These systems, devices, and methods reduce neurotoxin waste and treatment cost, as well as improving patient safety.

For example, disclosed embodiments comprise single-use packaging designed to contain a quantity of neurotoxin sufficient to treat a specific symptom, or to achieve a specific cosmetic or therapeutic treatment goal, such as a reduction in glabellar line visibility or migraine symptoms. Disclosed single-use packaging embodiments can also provide the clinician with visual cues to ensure the appropriate dosage for the desired treatment is administered to the patient.

Disclosed embodiments further comprise packaging vessels that reduce waste of neurotoxin formulations, for example by using a hydrophobic material or coating to reduce the “spread” and “adherence” of neurotoxin formulations inside a packaging vessel.

Further embodiments comprise packaging vessels shaped to concentrate neurotoxin formulations inside a packaging vessel.

### DETAILED DESCRIPTION

The present disclosure is directed toward systems and methods for reducing waste and expense of neurotoxin formulations while increasing patient safety.

### Definitions

“Administration,” or “to administer” means the step of giving (i.e. administering) a pharmaceutical composition or active ingredient to a subject. The pharmaceutical compositions disclosed herein can be administered via a number of appropriate routes, including oral, intramuscular, subdermal or subcutaneous routes of administration, such as by injection or use of an implant. Intramuscular injections can be, for example, superficial or intermediate.

“Botulinum toxin” or “botulinum neurotoxin” means a neurotoxin derived from Clostridium botulinum, as well as modified, recombinant, hybrid and chimeric botulinum toxins. A recombinant botulinum toxin can have the light chain and/or the heavy chain thereof made recombinantly by a non-Clostridial species. “Botulinum toxin,” as used herein, encompasses the botulinum toxin serotypes A, B, C, D, E, F, G and H. “Botulinum toxin,” as used herein, also encompasses both a botulinum toxin complex (i.e. the 300, 600 and 900 kDa complexes) as well as pure botulinum toxin (i.e. the about 150 kDa neurotoxic molecule), all of which are useful in the practice of the disclosed embodiments.

“Clostridial neurotoxin” means a neurotoxin produced from, or native to, a Clostridial bacterium, such as Clostridium botulinum, Clostridium butyricum or Clostridium beratti, as well as a Clostridial neurotoxin made recombinantly by a non-Clostridial species.

“Fixed-site, fixed-dose” (“FSFD”) means a treatment protocol that specifies the administration location(s) and unit dosage per location of a neurotoxin treatment

“Label” means any written, printed, or graphic matter on the container or package or any such matter clearly visible through the immediate carton, receptacle or wrapper.

“Neurotoxin” means a biologically active molecule with a specific affinity for a neuronal cell surface receptor. Neurotoxin includes Clostridial toxins both as pure toxin and as complexed with one or more non-toxin, toxin-associated proteins.

“Patient” means a human or non-human subject receiving medical or veterinary care.

“Pharmaceutical composition” means a formulation in which an active ingredient can be a Clostridial toxin. The word “formulation” means that there is at least one additional ingredient (such as, for example and not limited to, an albumin [such as a human serum albumin or a recombinant human albumin] and/or sodium chloride) in the pharmaceutical composition in addition to a botulinum neurotoxin active ingredient. A pharmaceutical composition is therefore a formulation which is suitable for diagnostic, therapeutic or cosmetic administration to a subject, such as a human patient. The pharmaceutical composition can be a lyophilized or vacuum dried condition, a solution formed after reconstitution of the lyophilized or vacuum dried pharmaceutical composition with saline or water, for example, or a solution that does not require reconstitution. As stated, a pharmaceutical composition can be liquid, semi-solid, or solid. A pharmaceutical composition can be animal-protein free.

“Purified botulinum toxin” means a pure botulinum toxin or a botulinum toxin complex that is isolated, or substantially isolated, from other proteins and impurities which can accompany the botulinum toxin as it is obtained from a culture or fermentation process. Thus, a purified botulinum toxin can have at least 95%, and more preferably at least 99% of the non-botulinum toxin proteins and impurities removed.

“Therapeutic formulation” means a formulation that can be used to treat and thereby alleviate a disorder or a disease and/or symptom associated thereof.

“Therapeutically effective amount” means the level, amount or concentration of an agent (e.g. such as a Clostridial toxin or pharmaceutical composition comprising Clostridial toxin) needed to treat a disease, disorder or condition without causing significant negative or adverse side effects.

“Treat,” “treating,” or “treatment” means an alleviation or a reduction (which includes some reduction, a significant reduction a near total reduction, and a total reduction), resolution or prevention (temporarily or permanently) of a symptom, disease, disorder or condition, so as to achieve a desired therapeutic or cosmetic result, such as by healing of injured or damaged tissue, or by altering, changing, enhancing, improving, ameliorating and/or beautifying an existing or perceived symptom, disease, disorder or condition.

“Unit” or “U” means an amount of active botulinum neurotoxin standardized to have equivalent neuromuscular blocking effect as a Unit of commercially available botulinum neurotoxin type A (for example, Onabotulinumtoxin A (BOTOX®)).

“Vessel” means a storage container for a pharmaceutical formulation, such as a vial.

### Single-Use Packaging

Disclosed embodiments comprise neurotoxin packaging systems and devices, for example vessels including vials, designed to minimize waste of neurotoxins. Disclosed embodiments provide packaging vessels designed for use with various neurotoxin treatments.

For example, FSFD treatment protocols employ a specific amount (typically expressed in units) of neurotoxin. Disclosed embodiments comprise vessels, for example vials, that contain that specific amount of neurotoxin.

Thus, in disclosed embodiments for use with a botulinum type A treatment for chronic migraine, 155 units of neurotoxin are packaged in a single vial. This provides the clinician with sufficient neurotoxin to treat the condition, without excess material that is either wasted or used in other treatments. In further embodiments designed for use in treatment of headache, such as migraine, the single vial can contain, for example, 140 units, 145 units, 150 units, 160 units, 165 units, 170 units, 175 units, 180 units, 185 units, 190 units, 195 units, 200 units, 205 units, 210 units, or the like. In further embodiments designed for use in treatment of overactive bladder, the single vial can contain, for example, 85 units, 90 units, 95 units, 100 units, 105 units, 110 units, 115 units, or the like.

In embodiments, the vial contains an additional amount of material to account for likelihood that not all of the neurotoxin formulation can be removed from the vial, as well as the “dead space” in the needle itself. For example, in embodiments, the vial contains an additional percentage of neurotoxin such as 5%, 10%, 15%, or the like. In embodiments, the vial contains an additional amount of neurotoxin, such as an additional 5 units, 10 units, 15 units, or the like.

Additional embodiments comprise visual cues to assist the practitioner in confirming the appropriate dose. For example, in embodiments for use in treatment of migraine, the stopper, the vial or the vial label or a portion thereof can be colored to distinguish the vial (and the amount of neurotoxin therein) from vials designed for use with other treatments.

For example, for a migraine treatment, the stopper, the vial, the vial label, or a portion thereof can be, for example, blue, while a stopper, vial, vial label, or a portion thereof for use in treating overactive bladder can be, for example, red. The specific color provides a visual cue for the clinician, confirming the appropriate dosage for the intended treatment. Therefore, the specific color used for a particular treatment vial is used to distinguish that vial from one used in a different treatment, and thus the actual colors of the individual vials can be determined by the formulation provider, and any color can be assigned to any treatment. Thus, in embodiments, the color of a stopper, vial, vial label, or a portion thereof can be, for example, red, orange, yellow, green, blue, indigo, violet, or combinations thereof.

### Single-Use Packaging Vessel Materials.

Disclosed embodiments also provide vessels with improved emptying characteristics. For example, current glass vials present a hydrophilic surface to an aqueous formulation such as a neurotoxin formulation. This type of surface at the formulation/vessel interface increases the spread of the aqueous neurotoxin formulation along the glass surface; in conventional glass pharmaceutical vials, 10-20% of the material typically sticks to the inner glass surfaces.

In contrast, disclosed vessels comprise a hydrophobic material that minimizes spread of the formulation. For example, disclosed vessels comprise a glass material comprising a hydrophobic material, such as a coating, along the inside surface of the vessel. In disclosed embodiments, the coating can be, for example, a chemically uniform coating such as SiO₂ or SiOₓC_{y}H_{z} that can act as a hydrophobic barrier to limit formulation spread along the interior surface of a vessel. Such a barrier can be deposited using plasma impulse chemical vapor deposition (PICVD) to apply several layers of silicon dioxide molecules (at a thickness of, for example, 100 to 200 nanometers) to the inside surfaces of glass vials. In embodiments, the SiOₓC_{y}H_{z} coating can be deposited from a hexamethyldisiloxane (HMDSO)/O₂ mixture in a parallel plate, capacitively-coupled, RF plasma reactor. In embodiments, the SiOₓC_{y}H_{z} coating can be applied at a thickness of, for example 20-40 nanometers.

In embodiments, the hydrophobic coating also reduces fogging during the lyophilization process and aids in maintaining a more cohesive “lyo cake,” which can increase cake stability during handling of vessels. This increase in stability can also improve “inspectability” of the lyophilized material, thus decreasing production costs.

Further disclosed embodiments comprise the use of hydrophobic materials to form the vessel itself. For example, in embodiments, the vessel is produced from Polytetrafluoroethylene (PTFE).

Drug manufacturers must overfill the vials to ensure delivery of the accurate dosage. In disclosed embodiments comprising a hydrophobic coating on the interior surface of the vessel, the overfill required is significantly reduced, leading to measurable cost savings.

Further disclosed embodiments comprise a vial “stopper” that comprises a hydrophobic material, for example a coating such as a silicon-based coating. In further embodiments, the stopper is made from a hydrophobic material, such as PTFE. The hydrophobic coating or material minimizes or prevents the adherence of the formulation to the stopper if the vial is inverted during or after reconstitution of the lyophilized formulation with saline. This in turn minimizes or prevents loss or waste of the formulation prior to administration to the patient.

Disclosed embodiments further comprise vessels, for example vials, comprising a “fill” line that provides an additional visual cue that the vessel has been filled with the appropriate amount of reconstitution fluid, for example saline.

### Single-Use Packaging Vessel Shapes

Disclosed embodiments further comprise neurotoxin formulation storage vessels shaped to minimize waste of the formulation. For example, disclosed embodiments comprise formulation vessels comprising an interior bottom that is sloped, for example toward one side of the vessel, to concentrate the formulation. Further embodiments comprise convex or concave interior bottom shapes to concentrate the formulation into a specific area or areas within the vessel.

In further embodiments, the vessel shape can be modified to lessen the distance between the opening of the vessel and the bottom of the vessel. For example, current BOTOX® vials have an aspect ratio (height:width) of roughly 2:1. In disclosed embodiments, this ratio is decreased, to produce a more “squat” vessel, such as one having an aspect ratio (height:width) of, for example, 1.9:1, 1.8:1, 1.7:1, 1.6:1, 1.5:1, 1.4:1, 1.3:1, 1.2:1, 1.1:1, 1:1, 0.9:1, 0.8:1, 0.7:1, 0.6:1, 0.5:1, 0.4:1, or the like. By decreasing the distance from the opening of the vessel to the bottom interior of the vessel, a shorter needle can be used, thus decreasing the “dead space” associated with the needle itself and reducing the amount of formulation needed to “prime” the needle before use.

Further embodiments comprise conical-shaped vessels, for example vessels with a wider base that tapers to a narrower opening.

### Single-Use Packaging Dilution Devices

Current methods of diluting neurotoxin formulations utilize an aqueous solution that is added to the neurotoxin vial. For example, BOTOX® is diluted with saline. However, managing dilution volumes can be problematic. Disclosed embodiments comprise pre-filled dilution devices, for example syringes, that contain adequate dilution volume to add to a vial to produce a neurotoxin formulation of a desired concentration. For example, disclosed dilution devices comprise syringes that contain a small excess amount of dilution material to account for the “dead space” in the syringe needle itself. For example, in disclosed embodiments, a prefilled syringe for use in diluting a 165 Unit vial can contain 3.35 mL of reconstitution fluid, for example saline, thus providing a diluent surplus of 50 μL to account for the dead space in the needle. Similarly, a prefilled syringe for use in diluting a 200 Unit vial can contain 4.05 mL of reconstitution fluid, for example saline, thus providing a diluent surplus of 50 μL to account for the dead space in the needle.

Additional embodiments comprise visual cues to assist the practitioner in confirming the appropriate dilution amount. For example, in embodiments for use in treatment of migraine, the stopper, the vessel (or a portion thereof) containing the dilution material can be colored to distinguish the vessel (and the amount of diluent therein) from vessels designed for use with other treatments.

For example, for a migraine treatment, the vessel (or a portion thereof) can be, for example, blue, while a vessel such as a vial or pre-filled syringe (or a portion thereof) for use in treating overactive bladder can be, for example, red. The specific color provides a visual cue for the clinician, confirming the appropriate dosage for the intended treatment. Therefore, the specific color used for a particular treatment vial is used to distinguish that vial from one used in a different treatment, and thus the actual colors of the individual vials can be determined by the formulation provider, and any color can be assigned to any treatment. Thus, in embodiments, the color of a stopper, vial, vial label, or a portion thereof can be, for example, red, orange, yellow, green, blue, indigo, violet, or combinations thereof.

In embodiments, the diluent vessel and the single-use packaging vessel employ the same color, providing the practitioner with visual cues ensuring that the appropriate amount of neurotoxin is diluted to the appropriate volume.

### Neurotoxin Compositions

Embodiments disclosed herein comprise neurotoxin compositions. Such neurotoxins can be formulated in any pharmaceutically acceptable formulation in any pharmaceutically acceptable form. The neurotoxin can also be used in any pharmaceutically acceptable form supplied by any manufacturer. Disclosed embodiments comprise use of Clostridial neurotoxins.

The Clostridial neurotoxin can be made by a Clostridial bacterium, such as by a Clostridium botulinum, Clostridium butyricum, or Clostridium beratti bacterium. Additionally, the neurotoxin can be a modified neurotoxin; that is a neurotoxin that has at least one of its amino acids deleted, modified or replaced, as compared to the native or wild type neurotoxin. Furthermore, the neurotoxin can be a recombinantly produced neurotoxin or a derivative or fragment thereof.

In disclosed embodiments, the neurotoxin is formulated in unit dosage form, for example, it can be provided as a sterile solution in a vial or as a vial or sachet containing a lyophilized powder for reconstituting in a suitable vehicle such as saline for injection.

In embodiments, the botulinum toxin is formulated in a solution containing saline and pasteurized Human Serum Albumin (HSA), which stabilizes the toxin and minimizes loss through non-specific adsorption. The solution can be sterile filtered (0.2 μm filter), filled into individual vials, and then vacuum-dried to give a sterile lyophilized powder. In use, the powder can be reconstituted by the addition of sterile unpreserved normal saline (sodium chloride 0.9% for injection).

In an embodiment, botulinum type A is supplied in a sterile solution for injection with a 5-mL vial nominal concentration of 20 ng/mL in 0.03 M sodium phosphate, 0.12 M sodium chloride, and 1 mg/mL HSA, at pH 6.0.

Although the composition may only contain a single type of neurotoxin, for example botulinum type A, disclosed compositions can include two or more types of neurotoxins, which can provide enhanced therapeutic effects of the disorders. For example, a composition administered to a patient can include botulinum types A and E. Administering a single composition containing two different neurotoxins can permit the effective concentration of each of the neurotoxins to be lower than if a single neurotoxin is administered to the patient while still achieving the desired therapeutic effects. This type of “combination” composition can also provide benefits of both neurotoxins, for example, quicker effect combined with longer duration.

The composition administered to the patient can also contain other pharmaceutically active ingredients, such as, protein receptor or ion channel modulators, in combination with the neurotoxin or neurotoxins. These modulators may contribute to the reduction in neurotransmission between the various neurons. For example, a composition may contain gamma aminobutyric acid (GABA) type A receptor modulators that enhance the inhibitory effects mediated by the GABA_{A} receptor. The GABA_{A} receptor inhibits neuronal activity by effectively shunting current flow across the cell membrane. GABA_{A} receptor modulators may enhance the inhibitory effects of the GABA_{A} receptor and reduce electrical or chemical signal transmission from the neurons. Examples of GABA_{A} receptor modulators include benzodiazepines, such as diazepam, oxaxepam, lorazepam, prazepam, alprazolam, halazeapam, chordiazepoxide, and chlorazepate. Compositions may also contain glutamate receptor modulators that decrease the excitatory effects mediated by glutamate receptors. Examples of glutamate receptor modulators include agents that inhibit current flux through AMPA, NMDA, and/or kainate types of glutamate receptors.

### Methods of Use

Disclosed neurotoxin compositions can be injected into the patient using a needle or a needleless device. In certain embodiments, the method comprises injecting the composition sub-dermally, subcutaneously, intramuscularly, or through superficial intramuscular injections, into the individual. For example, administering may comprise injecting the composition through a 27 gauge needle, 28 gauge needle, 29 gauge needle, 30 gauge needle, 31 gauge needle, 32 gauge needle, and/or a 33 gauge needle. In certain embodiments, the method comprises administering a composition comprising a botulinum toxin type A.

Administration of the disclosed compositions can be carried out by any suitable means, for example by syringe, catheters, needles and other means for injecting. The injection can be performed on any area of the mammal's body that is in need of treatment, however disclosed embodiments contemplate injection into the patient's head. The injection can be into any specific area such as epidermis, dermis, fat, muscle, nerve junction, or subcutaneous layer. Disclosed embodiments can comprise avoiding injecting certain areas, for example avoiding injecting at least one of or both of the frontalis and cervical paraspinal muscle regions.

More than one injection and/or sites of injection may be necessary to achieve the desired result. Also, some injections, depending on the location to be injected, may require the use of fine, hollow, Teflon®-coated needles, in certain embodiments guided, for example by electromyography.

The frequency and the amount of injection under the disclosed methods can be determined based on the nature and location of the particular area being treated. In certain cases, however, repeated injection may be desired to achieve optimal results. The frequency and the amount of the injection for each particular case can be determined by the person of ordinary skill in the art.

Although examples of routes of administration and dosages are provided, the appropriate route of administration and dosage are generally determined on a case by case basis by the attending physician. For example, the route and dosage for administration of a Clostridial neurotoxin according to the present disclosed invention can be selected based upon criteria such as the solubility characteristics of the neurotoxin chosen as well as the intensity and scope of the condition being treated.

Methods disclosed herein can comprise administration of a neurotoxin, for example a Clostridial toxin, for example a botulinum type A, to a patient to achieve a desired therapeutic or cosmetic goal. For example to prevent or alleviate the symptoms associated with chronic migraine. For example, disclosed methods can prevent or alleviate the occurrence of pain, nausea, vomiting, light sensitivity, sound sensitivity, and combinations thereof.

In embodiments, methods comprise administering a therapeutically effective amount of at least one neurotoxin to a nerve associated with at least one of the frontalis, nasalis, orbicularis oculi, corrugator, procerus, masseter, occipitalis, temporalis, and trapezius muscle regions. Further embodiments comprise the avoidance of injections to specific muscles. For example, disclosed methods comprise avoiding administration, for example injection, into the frontalis muscle, thus reducing the risk of ptosis. Further embodiments comprise avoiding administration, for example injection, into the cervical paraspinal muscles, thus reducing the risk of neck weakness.

### Neurotoxin Dosages

The neurotoxin can be administered in an amount of between about 10⁻³ U/kg and about 35 U/kg. In an embodiment, the neurotoxin is administered in an amount of between about 10⁻² U/kg and about 25 U/kg. In another embodiment, the neurotoxin is administered in an amount of between about 10⁻¹ U/kg and about 15 U/kg. In another embodiment, the neurotoxin is administered in an amount of between about 1 U/kg and about 10 U/kg. In many instances, an administration of from about 1 unit to about 300 Units of a neurotoxin, such as a botulinum type A, provides effective therapeutic relief. In an embodiment, from about 5 Units to about 200 Units of a neurotoxin, such as a botulinum type A, can be used and in another embodiment, from about 10 Units to about 100 Units of a neurotoxin, such as a botulinum type A, can be locally administered into a target tissue.

In embodiments, administration can comprise a total dose per treatment session of about 30 Units of a botulinum neurotoxin, or about 35 Units, or about 40 Units, or about 45 Units, or about 50 Units, or about 55 Units, or about 60 Units, or about 65 Units, or about 70 Units, or about 75 Units, or about 80 Units, or about 85 Units, or about 90 Units, or about 95 Units, or about 100 Units, or about 105 Units, or about 110 Units, or about 115 Units, or about 120 Units, or about 125 Units, or about 130 Units, or about 135 Units, or about 140 Units, or about 145 Units, or about 150 Units, or about 155 Units, or about 160 Units, or about 165 Units, or about 170 Units, or about 175 Units, or about 180 Units, or about 185 Units, or about 190 Units, or about 195 Units, or about 200 Units, or about 205 Units, or about 210 Units, or about 215 Units, or about 220 Units, or about 225 Units, or about 230 Units, or about 235 Units, or about 240 Units, or about 245 Units, or about 250 Units, or about 255 Units, or about 260 Units, or about 265 Units, or about 270 Units, or about 275 Units, or about 280 Units, or about 285 Units, or about 290 Units, or about 295 Units, or about 300 Units, or the like.

### EXAMPLES

The following non-limiting Examples are provided for illustrative purposes only in order to facilitate a more complete understanding of representative embodiments. This example should not be construed to limit any of the embodiments described in the present specification.

### Example 1

### Treatment of Chronic Migraine

A chronic migraine patient is treated via injection with 155 U of botulinum type A. To prepare for the injections, the clinician uses a 155 U neurotoxin migraine formulation vial, which is marked red to distinguish the migraine formulation vial from those intended for use with other treatments. The vial's interior surface is coated with a silicon-based formulation to provide hydrophobic properties. The bottom of the vial interior comprises a concave shape to reduce spread of the diluted formulation across the bottom. The stopper of the vial is made from a hydrophobic material, PTFE.

The clinician adds the proper dilution volume of saline to the red migraine formulation vial using a red prefilled migraine diluent syringe, to provide visual confirmation of the proper dilution for the intended treatment. The addition of the saline raises the volume level inside the vial to match a line on the vial itself, providing additional confirmation that the correct amount of diluent has been added to the vial.

The patient reports fewer migraines for 18 weeks following the treatment.

### Example 2

### Treatment of Chronic Migraine

A chronic migraine patient is treated via injection with 195 U of botulinum type A. To prepare for the injections, the clinician uses a 195 U neurotoxin migraine formulation vial, which is marked in pink to distinguish the migraine formulation vial from those intended for use with other treatments. The vial's interior surface is coated with a silicon-based formulation to provide hydrophobic properties. The bottom of the vial interior comprises a convex shape to reduce spread of the diluted formulation across the bottom. The stopper of the vial is pink, and made from a hydrophobic material, PTFE.

The clinician adds the proper dilution volume of saline to the pink migraine formulation vial using a pink prefilled migraine diluent syringe, to provide visual confirmation of the proper dilution for the intended treatment. The addition of the saline raises the volume level inside the vial to match a line on the vial itself, providing additional confirmation that the correct amount of diluent has been added to the vial.

The patient reports fewer migraines for 13 weeks following the treatment.

### Example 3

### Treatment of Chronic Migraine

A chronic migraine patient is treated via injection with 145 U of botulinum type A. To prepare for the injections, the clinician uses a 145 U neurotoxin migraine formulation vial, which is marked green to distinguish the migraine formulation vial from those intended for use with other treatments. The vial's interior surface is coated with a silicon-based formulation to provide hydrophobic properties. The bottom of the vial interior comprises a concave shape to reduce spread of the diluted formulation across the bottom. The stopper of the vial is green, and made from a hydrophobic material, PTFE.

The clinician adds the proper dilution volume of saline to the green migraine formulation vial using a green prefilled migraine diluent syringe, to provide visual confirmation of the proper dilution for the intended treatment. The patient is injected at several locations along the trigeminal nerve, including the frontalis, corrugator, procerus, and occipitalis.

The patient reports fewer migraines for 14 weeks following the treatment.

### Example 4

### Treatment of Chronic Migraine

A chronic migraine patient is treated via injection with 185 U of botulinum type A. To prepare for the injections, the clinician uses a 185 U neurotoxin migraine formulation vial, which is marked blue to distinguish the migraine formulation vial from those intended for use with other treatments. The vial's interior surface is coated with a silicon-based formulation to provide hydrophobic properties. The bottom of the vial interior comprises a concave shape to reduce spread of the diluted formulation across the bottom. The stopper of the vial is blue, and made from a hydrophobic material, PTFE.

The clinician adds the proper dilution volume of saline to the blue migraine formulation vial using a blue prefilled migraine diluent syringe, to provide visual confirmation of the proper dilution for the intended treatment. The patient is injected at several locations along the trigeminal nerve, including the corrugator, procerus, and occipitalis. The frontalis and the cervical paraspinal are not injected.

The patient reports fewer migraines for 16 weeks following the treatment.

### Example 5

### Treatment of Chronic Migraine

A chronic migraine patient is treated via injection with 150 U of botulinum type A. To prepare for the injections, the clinician uses a 150 U neurotoxin migraine formulation vial, which is marked yellow to distinguish the 150 U migraine formulation vial from those intended for use with other treatments. The vial's interior surface is coated with a silicon-based formulation to provide hydrophobic properties. The bottom of the vial interior comprises a concave shape to reduce spread of the diluted formulation across the bottom. The stopper of the vial is yellow, and made from a hydrophobic material, PTFE.

The clinician adds the proper dilution volume of saline to the yellow migraine formulation vial using a yellow prefilled migraine diluent syringe. A line on the inside of the vial provides visual confirmation of the proper dilution volume for the intended treatment.

The patient is injected at the upper frontalis (5 U in one intramuscular injection site on each side of the head at the hairline), the corrugator (5 U in one intramuscular injection site on each side of the head), the oculi (5 U in one intramuscular injection site on each side of the head), the temporalis (5 U per injection in three intramuscular injection sites on each side of the head anterior to the tragus), the occipitalis (10 U per injection in three intramuscular injection sites on each side of the head), and trapezius (5 U in one superficial intramuscular injection site on each side to the superior trapezius; 10 U in one intramuscular injection site on each side to the inferior trapezius). This provides a total of 150 U in 11 separate injection sites per side (22 injections total).

The nasalis, masseter, and cervical paraspinal muscles are not injected.

The patient reports fewer migraines for 16 weeks following the treatment.

### Example 6

### Treatment of Chronic Migraine

A chronic migraine patient is treated via injection with 195 U of botulinum type A. To prepare for the injections, the clinician uses a 195 U neurotoxin migraine formulation vial, which is marked orange to distinguish the 195 U migraine formulation vial from those intended for use with other treatments. The vial's interior surface is coated with a silicon-based formulation to provide hydrophobic properties. The bottom of the vial interior comprises a concave shape to reduce spread of the diluted formulation across the bottom. The stopper of the vial is orange, and made from a hydrophobic material, PTFE.

The clinician adds the proper dilution volume of saline to the 195 U orange migraine formulation vial using an orange prefilled migraine diluent syringe. A line on the inside of the vial provides visual confirmation of the proper dilution volume for the intended treatment.

The patient is injected at the upper frontalis (5 U in one intramuscular injection site on each side of the head at the hairline), the corrugator (5 U in one intramuscular injection site on each side of the head), the oculi (5 U in one intramuscular injection site on each side of the head), the temporalis (5 U per injection in three intramuscular injection sites on each side of the head anterior to the tragus), the nasalis (2.5 U per superficial intramuscular injection on each side of the head), the masseter (5 U in one intramuscular injection site on each side of the head), the occipitalis (10 U per injection in three intramuscular injection sites on each side of the head), and trapezius (5 U in one superficial intramuscular injection site on each side to the superior trapezius; 10 U in one intramuscular injection site on each side to the inferior trapezius). This provides a total of 195 U in 13 separate injection sites (26 injections total).

The cervical paraspinal not injected.

The patient reports fewer migraines for 16 weeks following the treatment.

### Example 7

### Treatment of Chronic Migraine

A chronic migraine patient is treated via injection with 155 U of botulinum type A. To prepare for the injections, the clinician uses a 155 U neurotoxin migraine formulation vial, which is marked in red to distinguish the 155 U migraine formulation vial from those intended for use with other treatments. The vial's interior surface is coated with a silicon-based formulation to provide hydrophobic properties. The bottom of the vial interior comprises a concave shape to reduce spread of the diluted formulation across the bottom. The stopper of the vial is red, and made from a hydrophobic material, PTFE.

The clinician adds the proper dilution volume of saline to the red migraine formulation vial using a red prefilled migraine diluent syringe. A line on the inside of the vial provides visual confirmation of the proper dilution volume for the intended treatment.

The patient is injected at the frontalis (5 U in two intramuscular injection sites on each side of the head), the corrugator (5 U in one intramuscular injection site on each side of the head), the procerus (5 U in one intramuscular injection site), the temporalis (5 U per injection in four intramuscular injection sites on each side of the head), the trapezius (5U in four intramuscular injection sites on each side of the head), and the cervical paraspinal group (5 U per injection in four intramuscular injection sites on each side of the head).

This provides a total of 155 U in 31 separate injection sites.

The patient reports fewer migraines for 16 weeks following the treatment.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present disclosure, which is defined solely by the claims. Accordingly, embodiments of the present disclosure are not limited to those precisely as shown and described.

Certain embodiments are described herein, comprising the best mode known to the inventor for carrying out the methods and devices described herein. Of course, variations on these described embodiments will become apparent to those of ordinary skill in the art upon reading the foregoing description. Accordingly, this disclosure comprises all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

Groupings of alternative embodiments, elements, or steps of the present disclosure are not to be construed as limitations. Each group member may be referred to and claimed individually or in any combination with other group members disclosed herein. It is anticipated that one or more members of a group may be comprised in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope otherwise claimed. No language in the present specification should be construed as indicating any non-claimed element essential to the practice of embodiments disclosed herein.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present disclosure so claimed are inherently or expressly described and enabled herein.

1) A vessel for neurotoxin formulations, wherein said vessel contains between 100% and 110% of the amount of neurotoxin intended to be administered to a patient in a single fixed-site fixed-dose treatment session.

2) The vessel of claim 1, wherein said treatment session comprises a migraine treatment.

3) The vessel of claim 2, wherein said migraine comprises chronic migraine.

4) The vessel of claim 3, wherein said vessel contains between 145 and 200 units of neurotoxin.

5) The vessel of claim 4, wherein said neurotoxin comprises a Clostridial toxin.

6) The vessel of claim 5, wherein said Clostridial toxin comprises a botulinum toxin.

7) The vessel of claim 6, wherein said botulinum toxin comprises botulinum type A, B, C, D, E, or F.

8) The vessel of claim 7, wherein said botulinum toxin comprises botulinum type A.

9) The vessel of claim 7, wherein said botulinum toxin comprises botulinum type B.

10) The vessel of claim 7, wherein said botulinum toxin comprises botulinum type C.

11) The vessel of claim 7, wherein said botulinum toxin comprises botulinum type E.

12) The vessel of claim 7, wherein said botulinum toxin comprises botulinum type F.

13) The vessel of claim 8, wherein said vessel contains 145 units of botulinum type A.

14) The vessel of claim 8, wherein said vessel contains 150 units of botulinum type A.

15) The vessel of claim 8, wherein said vessel contains 155 units of botulinum type A.

16) The vessel of claim 8, wherein said vessel contains 160 units of botulinum type A.

17) The vessel of claim 8, wherein said vessel contains 165 units of botulinum type A.

18) The vessel of claim 8, wherein said vessel contains 170 units of botulinum type A.

19) The vessel of claim 8, wherein said vessel contains 175 units of botulinum type A.

20) The vessel of claim 8, wherein said vessel contains 180 units of botulinum type A.

21) The vessel of claim 8, wherein said vessel contains 185 units of botulinum type A.

22) The vessel of claim 8, wherein said vessel contains 190 units of botulinum type A.

23) The vessel of claim 8, wherein said vessel contains 195 units of botulinum type A.

24) The vessel of claim 8, wherein said vessel contains 200 units of botulinum type A.

25) The vessel of claim 1, wherein said vessel further comprises a stopper.

26) The stopper of claim 25, wherein said stopper is color-coded for a specific treatment.

27) The color-coded stopper of claim 26, wherein said color comprises at least one of red, orange, yellow, green, blue, indigo, or violet, or combinations thereof.

28) The vessel of claim 1, wherein said vessel is color-coded for a specific treatment.

29) The color-coded vessel of claim 28, wherein said color comprises at least one of red, orange, yellow, green, blue, indigo, or violet, or combinations thereof.

30) A prefilled diluent syringe, wherein said prefilled diluent syringe contains between 100% and 110% of the amount of diluent intended to be used to dilute an amount of neurotoxin intended to be administered to a patient in a single fixed-site fixed-dose treatment session.

31) The prefilled diluent syringe of claim 30, wherein said diluent comprises saline.

32) The prefilled diluent syringe of claim 31, wherein said syringe is color-coded for a specific treatment.

33) The color-coded syringe of claim 32, wherein said color comprises at least one of red, orange, yellow, green, blue, indigo, or violet, or combinations thereof.

34) A kit comprising:
a vessel for a neurotoxin formulation, wherein said vessel contains between 100% and 110% of the amount of neurotoxin intended to be administered to a patient in a single fixed-site fixed-dose treatment session; and
a prefilled diluent syringe, wherein said prefilled diluent syringe contains between 100% and 110% of the amount of diluent intended to be used to dilute an amount of neurotoxin intended to be administered to a patient in a single fixed-site fixed-dose treatment session.

35) The kit of claim 34, wherein said treatment session comprises a migraine treatment.

36) The kit of claim 35, wherein said migraine comprises chronic migraine.

37) The kit of claim 34, wherein said vessel contains between 145 and 200 units of neurotoxin.

38) The kit of claim 37, wherein said neurotoxin comprises a Clostridial toxin.

39) The kit of claim 38, wherein said Clostridial toxin comprises a botulinum toxin.

40) The kit of claim 39, wherein said botulinum toxin comprises botulinum type A, B, C, D, E, or F.

41) The kit of claim 40, wherein said diluent comprises saline.

42) The kit of claim of claim 41, wherein said vessel is color-coded for a specific treatment.

43) The kit of claim 42, wherein said color comprises at least one of red, orange, yellow, green, blue, indigo, or violet, or combinations thereof.

44) The kit of claim 43, wherein said vessel comprises a stopper.

45) The kit of claim 44, wherein said stopper is color-coded for a specific treatment.

46) The kit of claim 45, wherein said color comprises at least one of red, orange, yellow, green, blue, indigo, or violet, or combinations thereof.

47) The kit of claim 34, wherein said vessel is comprised of a hydrophobic material.

48) The kit of claim 34, wherein said vessel comprises a hydrophobic coating on its interior surface.

49) The kit of claim 48, wherein said hydrophobic coating comprises Polytetrafluoroethylene (PTFE).

50). The kit of claim 44, wherein said stopper is comprised of a hydrophobic material.

51) The kit of claim 44, wherein said stopper comprises a hydrophobic coating on its outer surface.

52) The kit of claim 50, wherein said hydrophobic material comprises Polytetrafluoroethylene (PTFE).

53) The kit of claim 51, wherein said hydrophobic coating comprises Polytetrafluoroethylene (PTFE).

54) The kit of claim 34, wherein said vessel comprises a bottom interior that is not parallel to the bottom exterior.

55) The kit of claim 54, wherein said vessel bottom interior is sloped.

56) The kit of claim 54, wherein said vessel bottom interior is concave.

57) The vessel of claim 1, wherein said vessel comprises a bottom interior that is not parallel to the bottom exterior.

58) The vessel of claim 57, wherein said vessel bottom interior is sloped.

59) The vessel of claim 57, wherein said vessel bottom interior is concave.
